(19) United States
(12) Patent Application Publication
(10) Pub. No.: US 2017/0354600 A1
(43) Pub. Date: Dec. 14, 2017

(54) COMPOSITION AND METHOD FOR STABILIZING GSH

(71) Applicant: DENIS GUSTAVO BERNDT BRICENO, SANTIAGO (CL)

(72) Inventor: DENIS GUSTAVO BERNDT BRICENO, SANTIAGO (CL)

(21) Appl. No.: 15/619,848

(22) Filed: Jun. 12, 2017

Related U.S. Application Data
(60) Provisional application No. 62/348,286, filed on Jun. 10, 2016.

(51) Int. Cl. A61K 9/14; A61K 9/68; A23L 33/105; A61K 38/06

(52) U.S. Cl. CPC A61K 9/145; A61K 38/063; A61K 9/0058; A23L 33/105; A23V 2002/00

(57) ABSTRACT

A method for stabilize reduced glutathione using micronized curcumin, to provide a solid and liquid composition or formulation comprising a dispersion of the micronized curcumin. Also, a method for improving the oxidative stability of a peptide in a composition by adding micronized curcumin with the composition, and dispersing the micronized curcumin into the composition containing the peptide. The composition can include a chewing gum.

### CROSS-REFERENCE TO RELATED APPLICATIONS

This application claims the benefit of U.S. Provisional Application No. 62/348,286, filed Jun. 10, 2016, the disclosures of which are incorporated by reference in their entireties.

### BACKGROUND OF THE INVENTION

Reduced glutathione (GSH) is a tripeptide consisting of glycine, glutamic acid, and cysteine (Cys), with numerous biological functions in vivo. Aqueous solutions of GSH readily oxidize in air to form oxidized glutathione disulfide (GSSG) (See Biochemical Education vol. 11, 70 (1983)). Table 1 shows half-life values for GSH in different aqueous conditions.

TABLE 1

| | | | Half life |
|---|---|---|---|
| Conditions | | | (mins) |
| pH 6.5 | 20° C. | 0.1M K₂HPO₄/KH₂PO₄ Buffer | 16 |
| pH 7.5 | 20° C. | 0.1M K₂HPO₄/KH₂PO₄ Buffer | 9 |
| pH 8.5 | 20° C. | 0.1M K₂HPO₄/KH₂PO₄ Buffer | 1.3 |
| pH 8.5 | 0° C. | 0.1M K₂HPO₄/KH₂PO₄ Buffer | 8 |
| pH 8.5 | 40° C. | 0.1M K₂HPO₄/KH₂PO₄ Buffer | 0.2 |
| pH 8.5 | 20° C. | 0.1M K₂HPO₄/KH₂PO₄ Buffer + | 1.2 |
| | | 0.1 nM Cu + 2 | |
| pH 8.5 | 20° C. | 0.1M K₂HPO₄/KH₂PO₄ Buffer + | 70 |
| | | 1.0 nM EDTA | |

The oxidation of GSH to GSSG in in the presence of water and oxygen is a strong limitation for the use of GSH in food of pharmaceutical formulations.

Turmeric is the yellow colored powder rhizome of the perennial herb, Curcuma longa, which has been consumed by humans as a spice and medicinal agent for thousands of years. Curcumin is a yellow phenolic antioxidant isolated from turmeric. The chemical structure of curcumin in its enol and keto forms is illustrated below.

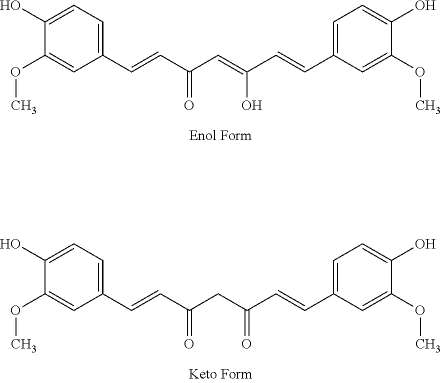 [SMILES: COc1cc(/C=C/C(=O)/C=C(O)/C=C/c2ccc(O)c(OC)c2)ccc1O ; COc1cc(/C=C/C(=O)CC(=O)/C=C/c2ccc(O)c(OC)c2)ccc1O]

Curcumin belongs to the group of diarylheptanoids found in various natural products. Curcumin extract can be purified from turmeric using different chemical and physical methods. A typical resulting product comprises crystals or granules of a high purity in the range of 75% to 98% by weight. Chinese application CN 201210064692 describes an extraction and purification method resulting in an extract in the form of crystals having a purity of 96% by weight (w/w) of curcumin, the disclosure of which is incorporated by reference in its entirety.

Curcumin is widely used as a natural colorant in many food and pharmaceutical formulations because of its high coloring capacity and high antioxidant capacity, resulting from its extensively conjugated double-bond structure.

Highly purified curcumin extracts are sparingly soluble in aqueous solution. For this reason, its bioavailability and coloring properties are limited because the size and morphology of the resulting crystals that are obtained from conventional purification methods. In order to improve the bioavailability and coloring properties of curcumin, several methods have been described in the prior art. For example, Japanese application JP2008-044524, the disclosure of which is incorporated by reference in its entirety, describes a method for improving water dispensability and light stability of curcumin extracts. The method includes the step of crushing curcumin-containing particles to a size of 210-420 nm in the presence of an emulsifier and an aqueous solvent.

Curcumin contains two electrophilic α,β-unsaturated carbonyl groups, which can react with nucleophilic compounds such as GSH. US Publication 2015/0320878 A1, the disclosure of which is incorporated by reference in its entirety, describes compositions and a method of preparation comprising a curcuminoid-peptide complex, including whey protein (WPI) and a list of peptides that may include GSH. The method comprises the steps of mixing the curcuminoid and the peptide in a solvent in order to create the curcuminoid-peptide complex, which is said to increase in the serum bioavailability of curcumin as compared to the ingestion of uncomplexed curcumin.

Although these methods and compositions of curcumin have been described, there still remains a need for an improved form of curcumin that is better able to stabilize compounds such as GSH.

### SUMMARY OF THE INVENTION

The present invention relates to an examination of the ability of curcumin to protect GSH, and other peptides, from oxidation, and a comparison against several known antioxidants in a hydrogen peroxide based oxidative model, which is a well-known powerful oxidant and a precursor of several free radical species such as peroxyl radical, hydroxyl radical, and superoxide, which are well known to affect GSH stability.

The present invention includes a composition comprising a quantity of curcumin consisting of dispersed particles having a micronized particle size. The particles of curcumin can have a particle size from 0.01 microns, and up to 5 microns, including a particle size up to 3 microns.

In another aspect of the invention, at least 50% by weight of the particles of curcumin have a particle size within the range of 0.8 microns to 1.2 microns, including at least 90% by weight of the particles of curcumin having a particle size within the range of 0.8 microns to 1.2 microns.

In a further aspect of the invention, the composition is selected from the group consisting of a chewing gum, a liquid composition, and a powdered or granular composition.

In a further embodiment of the invention, the composition comprising the quantity of curcumin consisting of dispersed particles having a micronized particle size, further includes a peptide that is mixed with the micronized curcumin. In one aspect of the invention, the peptide and the micronized curcumin material can be premixed. In other aspects of the invention, the peptide can be added to a composition comprising the micronized curcumin, or the micronized curcumin can be added to a composition comprising the peptide.

In an aspect of the invention, the peptide can include reduced glutathione (GSH).

The present invention includes a method for improving the anti-oxidation properties of curcumin, comprising the step of micronizing particles of curcumin to reduce the particle size of the curcumin to a target range limit sufficient to improve the anti-oxidant capacity of the curcumin.

The present invention further includes a method of improving the oxidative stability of a peptide in a composition, comprising the step of adding a micronized curcumin with the composition, and dispersing the micronized curcumin into the composition. In an aspect of the invention, the method further includes the step of combining the micronized curcumin with the peptide. In a further aspect, the micronized curcumin and peptide are pre-mixed prior to addition to the composition.

Despite the progress in the prior art in preparing particulate curcumin at submicron sizes, the effect of such particle size reduction on the antioxidant properties of curcumin extracts is unknown. Further, even though is known in the art that curcumin and GSH are able to react or form Curcumin-GSH adducts, due its chemical structure, the consequences of this reaction on the stability of GSH against oxidation is unknown.

The invention reveals that micronized curcumin can protect GSH from oxidation more effective, by one or more orders of magnitude, than other well-known antioxidant compounds tested, including conventional sources of powdered, non-micronized curcumin.

### DETAILED DESCRIPTION OF THE INVENTION

A composition that can be selected from the group consisting of a chewing gum, a liquid composition, and a powdered or granular composition, can comprise a quantity of micronized curcumin that is sufficient to provide an effective antioxidant properties to the composition for other components or ingredient in the composition, including proteins and peptides. A non-limiting example of a peptide is reduced glutathione (GSH). The micronized curcumin can consist of dispersed particles of curcumin having a micronized particle size.

The particles of curcumin can have a particle size from 0.01 microns, and up to about 5 microns, including up to about 3 microns. In an embodiment of the invention, the curcumin particle size includes a range generally centered around about 1 micron. One embodiment of a curcumin material can contain at least 50% by weight of the particles of curcumin having a particle size within the range of 0.8 microns to 1.2 microns, including at least 90% by weight of the particles of curcumin having a particle size within the range of 0.8 microns to 1.1 microns. Micronized curcumin is available from FMC Corporation in a 5% weight solution, marketed as Yellow ST 54; and in a 13% solution, marketed as Biokrum Yellow NB 8271.

The micronized curcumin can be provided as a powder or as a dispersed solution in a compatible liquid vehicle. Example compatible liquid vehicles include alcoholic base solvents or emulsifiers. A non-limiting example is polysorbate 80. The particle of curcumin can be mixed with particles or granules of other compounds, including other oxidant compounds and oxidizable compounds.

In order to evaluate the effectiveness of the method described in the present invention, 0.1 mmol of GSH was mixed with incremental concentrations of different food grade antioxidants (L-ascorbic acid, vitamin E (DL Alpha tocopheryl acetate), ascorbyl palmitate, vitamin A acetate, raw curcumin extract 96% w/w and micronized curcumin extract 5% w/w) in a solid or liquid composition and then exposed to oxidative conditions using hydrogen peroxide solution.

The antioxidant capacity is expressed as relative value 1/AC50, described as the magnitude corresponding to the inverse value of the antioxidant concentration expressed as mmol of antioxidant needed to prevent the oxidation of 50% of GSH present in a composition in a period of time of exposure to oxidation by hydrogen peroxide. A higher value of 1/AC50 correlates to a higher antioxidant capacity, and vice versa.

Antioxidant Test Materials
i) Reduced glutathione 96% was obtained from Kohjin Co. Ltd in the form of water soluble powder.
ii) Micronized curcumin, 5% (w/w), available from FMC Corporation in the form of a micronized liquid dispersion, having 90-95% by weight of curcumin particles with a size from 0.8 microns to 1.1 microns, and the balance of curcumin particles with a particle size from 1 micron to 3 microns.
iii) Raw curcumin extract, 96% (w/w), available from FMC Corporation in the form of a non-micronized powder, substantially having a particle size greater than 100 microns.
iv) Other test antioxidants:
   (1) L-Ascorbic acid (96% w/w) obtained from DSM in the form of water soluble crystals
   (2) Ascorbyl palmitate (96% w/w) obtained from DSM in the form of a water dispersible powder
   (3) Vitamin E as DL alpha tocopheryl acetate (50% w/w) obtained from DSM in the form of a water dispersible emulsion in powder form
   (4) Vitamin A acetate (50% w/w) obtained from DSM in the form of a water dispersible emulsion in powder form

### Example 1: Solid Composition

0.1 mmol of reduced glutathione (96%) was premixed with 5 g of liquid sorbitol (96%, Roquette Corp) using magnetic stirring for 15 minutes at room temperature. Then, as described in Table 2, an amount of each food grade antioxidant were separately added for each test, followed by an additional 10 minutes of stirring. The mixture was then homogeneously dispersed in 100 g of powder sorbitol using mechanical high shear blending for 3 minutes. 1 ml of hydrogen peroxide solution 1.0% w/v was added to each test and a continue mixing for additional 1 minute. A control test with no antioxidant was also performed.

TABLE 2
Different mmol antioxidant concentration
tested in solid composition

| Tested Compound | Antioxidant mmol | | | |
|---|---|---|---|---|
| L-Ascorbic Acid 96% | 0.001 | 0.01 | 1 | 10 |
| Vitamin E 50% | 0.001 | 0.01 | 1 | 10 |
| Ascorbyl palmitate | 0.001 | 0.01 | 1 | 10 |
| Vitamin A acetate 50% | 0.001 | 0.01 | 1 | 10 |
| Micronized Curcumin (5%) | 0.001 | 0.01 | 1 | 10 |
| Granular Curcumin 9 (6%) | 0.001 | 0.01 | 1 | 10 |

After 6 hours of exposure to oxidation, 5 g of the treated solid mixture was added to 30 ml of 0.1M disodium phosphate/0.001 mM EDTA pH 8.0 buffer solution. This solution was then extracted 3 times with chloroform using a separation funnel. The remaining aqueous phase was added to 100 ml volumetric flask and the volume was completed using 0.1M disodium phosphate/0.001 mM EDTA pH 8.5 buffer solution. The GSH present in this solution was quantify by colorimetric method at 405 nm using Elman's reagent, 5,5′-Dithio-bis-(2-nitrobenzoic acid), using L-glutathione 99%, provided by Sigma Aldrich, as standard. The retaining percentage for each solid composition was calculated and expressed as percentage values in Table 3.

TABLE 3
Retention percentage after 6 hours of oxidation in solid compositions

| Tested Compound | 0.001 mmol | 0.01 mmol | 18.18 mmol | 10 mmol |
|---|---|---|---|---|
| Ascorbic acid | 0.00% | 14.29% | 18.18% | 28.96% |
| Vitamin E | 24.16% | 31.04% | 39.61% | 45.19% |
| Micronized Curcumin | 32.73% | 38.70% | 54.55% | 80.78% |
| (5%) | | | | |
| Granular Curcumin | 27.12% | 31.65% | 42.23% | 49.16% |
| (96%) | | | | |
| Vitamin A | 28.57% | 33.77% | 46.36% | 51.04% |
| Ascorbyl palmitate | 18.18% | 25.97% | 36.88% | 46.10% |

Control no antioxidants: 12.0% after 6 hours

The results of antioxidant capacity are expressed as relative value 1/AC50 6 h in Table 4. 1/AC50 6 h is the magnitude corresponding to the inverse value of the antioxidant concentration expressed as mmol of antioxidant needed to prevent the oxidation of 50% of GSH in a period of time of 6 hours exposed to oxidation by hydrogen peroxide. A higher value of 1/AC50 6 h means a higher antioxidant capacity and vice versa. For those 1/AC50 6 h values that were not found directly on the mmol concentration tested, an estimation using linear regression was performed in order to determine those values.

TABLE 4
Antioxidant capacity (1/AC50 6 h) in solid compositions

| Antioxidant Compound | 1/AC50 6 h |
|---|---|
| Ascorbic acid | 0.05 |
| Vitamin E | 0.078 |
| Micronized Curcumin (5%) | 1 |
| Granular Curcumin (96%) | 0.105 |
| Vitamin A | 0.12 |
| Ascorbyl palmitate | 0.1 |

### Example 2: Liquid Compositions

0.1 mmol of reduced glutathione (Kohjin Co. Ltd) was premixed with 5 g of liquid sorbitol (96%, Roquette Corp) using magnetic stirring for 10 minutes. Then, as described in Table 5, incremental concentrations of food grade antioxidants where added followed by additional 10 minutes of stirring. The mixture was then added to 100 g of 0.1M disodium phosphate/0.001 mM EDTA pH 8.0 buffer solution, then 1 ml of hydrogen peroxide solution 0.5% w/v was added to each test. A control test with no antioxidant was also performed.

TABLE 5
Different mmol antioxidant concentration
tested in liquid composition

| Tested Compound | Antioxidant mmol | | | |
|---|---|---|---|---|
| L-Ascorbic Acid | 0.001 | 0.01 | 1 | 10 |
| Vitamin E | 0.001 | 0.01 | 1 | 10 |
| Ascorbyl palmitate | 0.001 | 0.01 | 1 | 10 |
| Vitamin A acetate | 0.001 | 0.01 | 1 | 10 |
| Curcumin 5% | 0.001 | 0.01 | 1 | 10 |
| Curcumin 96% | 0.001 | 0.01 | 1 | 10 |

After 1 hour of exposure to oxidation, 5 grams of the treated solution was added to 30 ml of 0.1M disodium phosphate/0.001 mM EDTA pH 8.5 buffer solution. This solution was then extracted 3 times with chloroform using a separation funnel. The remaining aqueous phase was added to a 100 ml volumetric flask and the volume was completed using 0.1M disodium phosphate/0.001 mM EDTA pH 8.0 buffer solution. The GSH present in this solution was quantify by colorimetric method at 405 nm using Elman's reagent, 5,5′-Dithio-bis-(2-nitrobenzoic acid), using L-glutathione 99%, provided by Sigma Aldrich, as standard. The retaining percentage for each solid composition was calculated and expressed as percentage values in Table 6.

TABLE 6
Retention percentage after 1 hour of oxidation in liquid composition

| Tested Compound | 0.001 mmol | 0.01 mmol | 1 mmol | 10 mmol |
|---|---|---|---|---|
| Ascorbic acid | 0.00% | 0.00% | 17.92% | 32.08% |
| Vitamin E | 10.39% | 16.88% | 30.39% | 41.69% |
| Micronized Curcumin | 20.78% | 31.17% | 52.21% | 61.04% |
| (5%) | | | | |
| Granular Curcumin | 13.74% | 18.56% | 40.01% | 44.93% |
| (96%) | | | | |
| Vitamin A | 14.29% | 21.95% | 33.25% | 49.79% |
| Ascorbyl palmitate | 0.00% | 13.38% | 18.83% | 36.36% |

The results of antioxidant capacity are expressed as relative value 1/AC50 1 h in Table 7. 1/AC50 1 h is the magnitude corresponding to the inverse value of the antioxidant concentration expressed as mmol of antioxidant needed to prevent the oxidation of 50% of GSH in a period of time of 1 hour exposed to oxidation by hydrogen peroxide. A higher value of 1/AC50 1 h means a higher antioxidant capacity and vice versa. For those 1/AC50 1 h values that were not found directly on the mmol concentration tested, an estimation using linear regression was performed in order to determine those values.

TABLE 7
Antioxidant capacity (1/AC50 1 h) in liquid compositions

| Antioxidant Compound | 1/AC50 1 h |
|---|---|
| Ascorbic acid | 0.02 |
| Vitamin E | 0.08 |
| Curcumin 5% | 1 |
| Curcumin 96% | 0.086 |
| Vitamin A | 0.1 |
| Ascorbyl palmitate | 0.06 |

Unexpectedly and unpredictably, it was found that micronized curcumin had by far the highest 1/AC50 1 h value in all the oxidative models tested. Compare to raw non-micronized curcumin and other antioxidants, the ability of micronized curcumin to prevent GSH oxidation was significantly improved in solid and liquid compositions. The present invention may be useful for those applications in which GSH has limitations since its inherent instability to oxidation.

### Example 3: Chewing Gum Composition Containing GSH and Curcumin

The following chewing gum composition was prepared containing GSH and curcumin, according to the formulation shown in Table 8.

TABLE 8
Chewing gum base formulation

| Ingredient | mg | Percentage |
|---|---|---|
| Gum base | 500 | 45.45% |
| Sorbitol Powder | 272.7 | 24.79% |
| Sunflower lecithin | 55 | 5.00% |
| Vegetable oil | 11 | 1.00% |
| liquid sorbitol | 55 | 5.00% |
| Glycerin | 22 | 2.00% |
| Mannitol | 60 | 5.45% |
| Curcumin dispersion 5% | 7.7 | 0.70% |
| Mint Flavor liquid | 20 | 1.82% |
| Menthol | 10 | 0.91% |
| Softener | 10 | 0.91% |
| Sucralose | 6.6 | 0.60% |
| Encapsulated peppermint flavors (10%) | 20 | 1.82% |
| Encapsulated sucralose (10%) | 20 | 1.82% |
| L-Glutathione | 30 | 2.73% |
| Total | 1100 | 100.00% |

In a chewing gum double-sigma blade kneader pre-equilibrated to a temperature of 50° C., the amount of gum base provided by Gum Base Co., of the formula was melted at 50° C., and 50% of the sorbitol powder and the mannitol powder (both products provided by Roquette Corporate) were added to the kneader, and mixed for 10 minutes with the melted gum base. Once the previous mixture was homogeneous, the glycerol, sunflower lecithin and vegetable oil were added and after a further 10 minutes, the remaining 50% of the sorbitol powder and the total sucralose was added and mixed for 10 minutes. After this mixture was homogeneously mixed, the temperature was reduced to 35° C. by using a cooling jacket. Then the flavors, softener, menthol, encapsulated flavors and menthol were added and mixed by 5 minutes to form a chewing gum base.

In a separate stainless steel recipient (container), curcumin was dispersed in liquid sorbitol at room temperature for 10 minutes, using magnetic stirring until completely incorporated into the sorbitol. Subsequently the amount of L-glutathione was added and mixed for an additional 15 minutes using mechanical stirring. Once homogeneous, this mixture was added to the chewing gum base and mixed for additional 10 minutes.

The stabilization method for GSH comprising the premixing curcumin with said GSH, resulted in a chewing gum with a vibrant yellow color. The Hunter LAB values for said chewing gum were: L: 75.13; a: −5.54; b: 76.15.

By comparison, a composition having an equimolar concentration of granular, non-micronized curcumin has a much weaker color or hue.

1. A composition comprising a quantity of curcumin consisting of dispersed particles having a micronized particle size.

2. The composition of claim 1, wherein the particles of curcumin have a particle size from 0.01 microns, and up to 5 microns.

3. The composition of claim 1, wherein the particles of curcumin have a particle size up to 3 microns.

4. The composition of claim 2, wherein at least 50% by weight of the particles of curcumin have a particle size within the range of 0.8 microns to 1.2 microns.

5. The composition of claim 4, wherein at least 90% by weight of the particles of curcumin have a particle size within the range of 0.8 microns to 1.2 microns.

6. The composition of claim 1, wherein at least 50% by weight of the particles of curcumin have a particle size within the range of 0.8 microns to 1.2 microns.

7. The composition of claim 6, wherein at least 90% by weight of the particles of curcumin have a particle size within the range of 0.8 microns to 1.2 microns.

8. The composition of claim 1, where the composition is selected from the group consisting of a chewing gum, a liquid composition, and a powdered or granular composition.

9. The composition of claim 1, further including a peptide that is mixed with the micronized curcumin.

10. The composition of claim 9, where the peptide includes reduced glutathione (GSH).

11. The composition of claim 4, further including a peptide that is mixed with the micronized curcumin.

12. The composition of claim 11, where the peptide includes reduced glutathione (GSH).

13. A method for improving the anti-oxidation properties of curcumin, comprising the step of micronizing particles of curcumin to reduce the particle size of the curcumin to a target range limit sufficient to improve the anti-oxidant capacity of the curcumin.

14. A method of improving the oxidative stability of a peptide in a composition, comprising the step of adding a micronized curcumin with the composition, and dispersing the micronized curcumin into the composition.

15. The method of claim 14 further including the step of combining the micronized curcumin with the peptide.

16. The method of claim 15 where the micronized curcumin and peptide are premixed prior to addition to the composition.

17. The method of claim 16, where the peptide includes reduced glutathione (GSH).
